(19) United States
(12) Patent Application Publication
(10) Pub. No.: US 2004/0185467 A1
(43) Pub. Date: Sep. 23, 2004

(54) Genotyping

(76) Inventors: Lawrence M. Kauvar, San Francisco, CA (US); Magnus Strandh, San Francisco, CA (US); Remy Cromer, Saratoga, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/713,632

(22) Filed: Nov. 13, 2003

Related U.S. Application Data
(60) Provisional application No. 60/426,782, filed on Nov. 14, 2002.

(51) Int. Cl. C12Q 1/68

(52) U.S. Cl. 435/6

(57) ABSTRACT

Improved methods using microscope based detection for identifying nucleic acid polymorphisms rely on localizing the region to be tested using particulate labels. The length of a segment containing tandem repeats can be determined by the intensity of signal emitted by a fluorophore associated with the repeat and bracketed by the labels; the presence of an allele containing a restriction site can be identified by viewing the association or dissociation of particulate labels. Single nucleotide polymorphisms characterized by the presence or absence of a referent base can be detected with a large differential in binding energy using short probes, by virtue of localizing the base to be interrogated with flanking particulate labels. The methods may be performed on a multiplicity of test nucleic acids simultaneously by employing a multiplicity of particulate labels with differing hues.

### TECHNICAL FIELD

The invention relates to methods to establish genetic fingerprints or to characterize individual chromosomal sections. More precisely, the invention concerns the use of multihued beads and color intensity to detect single nucleotide polymorphisms and analogs of restriction fragment length polymorphisms.

### BACKGROUND ART

Many approaches have been used to characterize DNA samples for identity and forensic purposes, for example. One particularly common technique is to determine the pattern of sizes of restriction fragments that result when DNA samples are treated with one or more restriction enzymes. The pattern of sizes can be determined using standard electrophoretic techniques. In addition, single nucleotide polymorphisms (SNPs) can be detected using probes of various descriptions. The present methods to determine size patterns or the presence of SNPs generally require amplification of DNA samples using, for example, the polymerase chain reaction (PCR) or an alternative method of providing sufficient copies of the DNA segment containing the base to be interrogated or the fragment to be analyzed to carry out this determination on the semi-macroscopic scale used in commonly available methods.

Forensic DNA testing, in particular, often must be conducted on trace quantities of DNA. Extensive amplification by PCR is undesirable because “ghost” DNA (trace contaminants in the lab) can also be amplified, imposing a significant quality control burden. An assay that is amenable to trace DNA with little or no PCR amplification is therefore especially useful in this context. Further, a method that does not require any physical separation of the sample reduces the chances for mislabeling of the samples during transfer from source to the separation device to the reader. Reduced handling also speeds up the process.

Prior methods for interrogating a single base include: (i) hybridization of the target DNA fragment to an array of oligonucleotides that differ in the SNP position, with differential extent of binding indicating which alternative is contained in the probe; (ii) extension of a primer across the SNP site with a chain terminating dideoxynucleotide specific for one allele causing a shorter chain to form than for the other allele, with length determined by mass spectroscopy; and (iii) creation of a ligatable junction at the site of the SNP, with ligation dependent on the correct allele being present to form a base pair at the junction. In the first of these instances, the detection is based on the few kilocalories differential in stability of an oligonucleotide that does or does not include a single mismatch. The use of proteins (restriction enzyme, polymerase, or ligase) amplifies that small differential. The use of multiple related probe sequences likewise amplifies the signal by allowing signal averaging. The present invention provides convenient methods to overcome the smallness of energy differential that is associated with a single mismatch.

The feasibility of detecting single nucleotide variants in a single copy of genomic DNA (metaphase chromosome spread) has been reported using “padlock” probes, which circularize by DNA ligation when hybridized to an appropriate target DNA, with a sandwich of amplification fluors added onto the padlock for detection (Antson, D., et al., Nucleic Acids Research (2000) 28(12):e58).

PCT publication WO 00/14545 published 16 Mar. 2000 describes methods for preparing multihued labels which can be identified directly using microscope based techniques. By using various combinations and ratios of signal-generating moieties attached to a particulate support, “beads” with multiple hues can be generated. The beads may also be attached to a reagent specific for an intended target which permits their use as labels to identify or quantitate the target. The contents of this document are incorporated herein by reference.

As set forth in the above mentioned document, these multihued labels are particularly useful for observing specific moieties by wide field or confocal microscopy. By the use of these labels, intracellular activities, for example, can be monitored. A particularly useful type of microscope for observing such events is that described in PCT publication WO 00/19262 published 6 Apr. 2000, issued as U.S. Pat. No. 6,444,992, and also incorporated herein by reference. This microscope permits detection of individual hues by employing multiple detectors sensitive to different wavelengths and ordering the images in accordance with the intensities of the various wavelengths.

Prior use of multihued beads for DNA analysis has employed formats in which a single bead is analyzed, in contexts other than observation under a microscope. For example the Luminex system for bead detection by flow cytometry has been used to type bacterial species (Ye, F., et al., Human Mutation (2001) 17:305-316).

Lu, M., et al., Human Mutation (2002) 19:416-422) describe an assay in which a triplex DNA structure is formed if the wild type base is present, followed by restriction cleavage which dissociates a fluor from a fluorescent quencher thereby generating a signal.

The present invention takes advantage of the techniques described in the aforementioned WO 00/14545, WO 00/19262 and U.S. Pat. No. 6,444,992 and permits analysis of DNA on a microscopic scale without the necessity for amplification reactions or physical separation steps, including washing steps. Additional tools related to these goals are also disclosed. Microscopy allows visualization of pairs of beads, which enables higher degrees of multiplexing, increased specificity, and inclusion of internal calibration controls.

### DISCLOSURE OF THE INVENTION

The present invention is directed to nucleic acid analysis, in particular genomic DNA analysis, taking advantage of the capacity to detect even a single copy of a nucleic acid microscopically if it is labeled with a sufficiently bright fluor such as a microsphere of 20 nm diameter or larger, or a single quantum dot.

Briefly, the invention employs identification probes which are oligomers coupled to particulate labels that can be directly observed by microscopy. The identification probes are hybridized to a test nucleic acid to determine the presence of the test nucleic acid or to identify and locate a region that can be interrogated for the number of tandem repeats in a stretch of repetitive regions or which can be interrogated for the presence or absence of a single nucleotide polymorphism (SNP). Typically, two such identification probes are used to bracket the region to be interrogated; however, in some embodiments, a single identification probe can be used if desired. By ascertaining the presence of the test nucleic acid or by identifying the location of the interrogated region by this method, which is directly observable using microscope-based techniques, the necessity to amplify the target DNA can be avoided and relatively simple methods to assay the region to be interrogated can be used.

For example, in one embodiment, the invention method measures the number of copies of a repeating unit length “repetitive element” nucleotide sequence at a locus defined by flanking identification probes by measuring the intensity of signal emitted by labels specific to the repeating unit. Such variable number tandem repeat (VNTR) polymorphisms are the most widely used parameters for forensic DNA work. At a given chromosomal site, the number of copies of the simple repeat typically ranges from 3-30. Any given individual shows two alleles (maternally and paternally derived) which can be the same or different. The Federal Bureau of Investigation typically uses 13 loci to achieve odds of a coincidental match to below 1/100 billion; the present invention is particularly useful in streamlining the determination of VNTR polymorphisms as further described below.

The present invention also detects the presence of single nucleotide polymorphisms (SNPs) which are related to restriction sites by detecting the ability of the appropriate restriction enzyme to dissociate labels on the identification probes bracketing the location of the SNP. SNPs in general may also be detected by directly visualizing the polymorphic base in situ.

Thus, in one aspect, the invention is directed to a method to obtain data equivalent to electrophoretic visualization of restriction fragment length polymorphisms arising from variable numbers of tandem repeats of a repetitive DNA element, such as the Alu sequence, which method comprises coupling an identification probe comprising first particulate label upstream (i.e., 5′) of said segment and a second identification probe comprising second particulate label downstream (i.e., 3′) of said segment and coupling each repeating sequence in the bracketed segment to a signal generating moiety of predetermined intensity. Then the total intensity of the signal generating moiety associated with said first and second particulate label is observed. The intensity of the signal is proportional to the number of tandem repeats. The location of the repeat is identified by the bracketing probes. The same Alu probe can thus be applied to the Alu repeats at multiple loci without the signals from different loci adding, due to the readout being restricted to the DNA bracketed by the identification probes. Following the completion of the human genome sequence, such flanking identification probes are now readily prepared.

The following two aspects of the present invention relate to the determination of single nucleotide polymorphisms (SNPs); these approaches are alternative methods for interrogating a single base within a sequence.

In one aspect related to SNP detection, the invention is directed to a method which applies when the presence of a particular base at a site interrogated results in the presence of a restriction site in double-stranded DNA. This method comprises reacting a single-stranded DNA to be tested for the presence of the SNP with a first oligonucleotide which oligonucleotide contains a first restriction site haploid and is complementary to the portion of the test DNA which would contain the SNP. The SNP locus comprises a potential second restriction site haploid complementary to the first. The first oligonucleotide is coupled to a first particulate label. The tested DNA is also reacted with a second oligonucleotide coupled to a second particulate label, which second nucleotide is complementary to a portion of the tested DNA contiguous with the locus of said possible SNP. Thus, double-stranded nucleic acid is obtained wherein the first restriction site haploid in said first oligonucleotide is in a position complementary to the locus of the possible SNP. The resulting double-stranded DNA is contacted with a restriction enzyme which cleaves at the restriction site formed when a locus complementary to the first oligonucleotide is present and the association or dissociation of said first and second particulate label is then observed. Continued association of said first and second particulate labels indicates the absence of the complement to said first restriction site haploid and dissociation of said first and second particulate label indicates the presence of the complement to said restriction site haploid. Direct visualization of upstream and downstream beads simplifies the assay over that described by Lu, M., et al. (supra), while increasing specificity.

In an alternative aspect of SNP detection, the invention is directed to methods to detect a single nucleotide polymorphism which does not require the presence of a restriction site at the location of the altered base. In one such method, as in the method described above, identification probes, which comprise labeled beads associated with oligomers that bind specifically upstream and downstream of the putative single nucleotide polymorphism site are employed to bracket the site. Then, an assay probe, which is a small, e.g., 5-mer, oligonucleotide labeled with a bright fluorescent label is used as a probe for the possibly modified base. As only a short oligomer is used, there is sufficient energy difference between a complete match and a single base pair mismatch to provide effective differentiation in binding. The presence of the assay probe associated with the beads indicates the presence, at the interrogated locus of a base complementary to that in the assay probe position. That is, the specificity with regard to chromosomal locus is established by the identification probes independently of the specificity with regard to the SNP assay probe itself, thus amplifying the differential binding of the short SNP assay probe. The fact that such a short oligonucleotide will bind to numerous sites in the genome is irrelevant, because only the bracketed locus is examined.

In another method to detect a SNP not requiring a potential restriction site, a single-stranded target is interrogated by a method which comprises hybridizing to the target nucleotide sequence an identification probe which is a bead-labeled primer that terminates immediately adjacent to the base to be interrogated. The reaction mixture further contains an assay probe such as a termination oligomer that comprises a dideoxynucleotide or other terminator which can be incorporated by virtue of its complementarity to a referent base in the SNP. The terminator is coupled to a linker that effects a “handle” such that an additional portion of the assay probe (such as additional nucleotides in this termination oligomer) are offset from the target nucleic acid. These offset nucleotides have a predetermined sequence designed to bind to still another oligonucleotide “key” that contains a fluorescent label such as a quantum dot. Alternatively, if the additional portion of the assay probe is other than a nucleotide sequence, it will include a member of a specific binding pair which offers a binding site for a “key” which contains a fluorescent label coupled to the opposite member of the specific binding pair, or it will, itself, include a fluorophore. The functions of the handle are to minimize inhibition of the polymerase and to avoid steric hindrance for the key to bind. If the terminator is incorporated, the assay probe is coupled to the target; if the terminator nucleotide is not incorporated, the assay probe is not. The incorporation can be detected by hybridizing the offset extended portion of the termination oligomer to a complementary key which is coupled to the fluorescent marker; more generically, the incorporation of an assay probe in general can be detected by coupling the offset extended portion of the probe which contains the binding pair member to a key which contains the fluorescent marker coupled to the complementary binding pair member. Alternatively, the assay probe may itself contain a fluorescent marker in the extended portion. Preferably, in addition to the primer labeled with bead that has been coupled upstream of the interrogated base, an additional identification marker, i.e., oligomer complementary to the sequence downstream of the base to be interrogated and coupled to a bead, is supplied as well, thus conferring additional specificity on the location of the incorporated assay probe. The incorporation of the assay probe is detected by the association of the labeled assay probe or labeled key with the first (and, if applicable, second) bead associated with the identification probe.

This variation has the advantage that only four different assay probes need be employed to interrogate any target nucleic acid; when the prior described standard 5-mer assay probe is employed, a variety of probes would need to be provided depending on the surroundings of the base to be interrogated.

The components in each of the foregoing methods can be packaged into kits for the convenience of the user. Typically, any identification probes, assay probes, detection probes, or other reagents, such as polymerase, required to carry out the methods of the invention may be included in separate containers and packaged in quantities suitable for carrying out the methods. The kit will also include instructions for carrying out the methods. Since the identification probes are attached to beads that can be prepared in many distinguishable types, a correspondingly large number of loci can be interrogated concurrently.

### BRIEF DESCRIPTION OF THE DRAWINGS

FIGS. 1-3 are diagrams of the methods of the invention including schematic representations of the patterns observed microscopically.

FIG. 1 illustrates hybridization of an assay probe for the repetitive element A/u between flanking sites to which beads are bound.

FIG. 2 illustrates dissociation of beads from a DNA fragment upon restriction enzyme cleavage of DNA between the flanking sites to which the beads included in identification probes are bound.

FIG. 3 illustrates use of an assay probe comprising a terminator and an offset extended portion or “handle” (zzzz) to facilitate hybridization of a detector “key” moiety (yyyy) at a putatively polymorphic site between flanking sites to which beads incorporating identification probes are bound.

### MODES OF CARRYING OUT THE INVENTION

The present invention offers highly sensitive ways to analyze a target nucleic acid without the need for physical separation steps. The target nucleic acid can be DNA, RNA or variations thereof including peptide nucleic acid (PNA); nucleic acids with alternative linkages, and the like. Of most practical interest, however, is genomic DNA which is widely analyzed for establishing identity and other inheritable features. While the invention is particularly useful for establishing genetic identity by virtue of providing characteristic patterns derived from the genomic DNA of individuals, its applications are not limited to this particular use. It may be of interest, for example, to determine the presence or absence of a particular SNP which may have been shown associated with a particular condition or to determine the length of a particular genetic repeat which may also be associated with a specific condition such as Huntington's disease. In these instances, the “pattern” which emerges is much simpler—the invention method can determine, specifically, each of these parameters on an appropriate portion of DNA. Other applications will become apparent as need for them arises. The basic steps in the invention method can be performed on a range of test samples with a range of desired outcomes. In particular, multiplexing is readily implemented. In all cases, specificity for the genetic locus is established by identification probes which are oligomers coupled to beads, allowing simplified methods to be used to characterize the nature of the DNA at that locus. The high sensitivity for detection, and susceptibility to multiplexing, of the beads facilitates the analysis.

As used herein, “nucleic acid” and “oligonucleotide” or “oligomer” refer to DNA, RNA, peptide nucleic acid, DNA or RNA having alternative linkages to phosphodiesters—e.g., thioester linkages, phosphoramidite linkages, and the like as well as these compounds which have modified forms of the naturally occurring bases. The oligonucleotides useful in the invention are designed so as to specifically bind sequences in a target nucleic acid; as long as sufficient complementarity or binding specificity is obtained, the nature of the linkages of individual units and the precise nature of the bases contained in these units is unimportant. Of course, the simplest embodiments are those wherein DNA, RNA, linkage modified forms of these nucleic acids or nucleotides contain sequences of the naturally occurring adenine, cytosine, thymine, guanine and uracil are employed. Typically, the target nucleic acid will be DNA, or less frequently RNA, and the oligonucleotide (or “oligomer”) is more variable in terms of backbone linkages and coupled bases.

All of the invention methods employ “beads” that are particulate labels coupled usually to oligomers, but more generally to one member of a specific binding pair [such as biotin/avidin or antibody/antigen (or complementary nucleotide sequences)]. The particulate labels are preferably solid supports to which signal generating moieties are conjugated; in the method of the invention, fluorophores are preferred as they are directly observable using microscopic techniques. Suitable labels are described in the above-referenced PCT publication WO 00/14545; typically, the particles range in size from about 20 nm to about 5 μm; preferred particles are in the range of 100-1000 nm. These particles are readily observable by microscopy. The particles can be formed of any suitable support such as latex, polystyrene, silica, and the like. The fluorophores are also variable and include, for example, fluorescein, green fluorescent protein (in various colors), dansyl, Texas red, merocyanines, quantum dots and the like. A wide variety of such fluorophores and dyes is available commercially. The fluorophore or other signal generating moiety is coupled to the particulate support using standard linking or entrapment technology appropriate to the choice of support and signal generating moiety. Similarly, this particulate label is coupled to the oligomer or other specific binding pair member using standard linking technology including the use of commercially available linkers or biospecific bridging elements such as avidin-biotin. These methods are well known and understood by practitioners in the art of biospecific binding assays.

As noted above, the “identification probes” used to identify a particular locus for testing comprise oligomers that hybridize to specific regions of target nucleic acid, wherein said oligomers are coupled to the labeled beads. Typically, the oligomers are designed to hybridize to a known particular region and are sufficient length to confer the requisite specificity. It has been calculated that the human genome and genomes of most higher organisms do not exhibit redundancy within themselves over regions of at least 18 nucleotides. Thus, if a single bead-linked oligomer were to be used to identify a region of the genome, typically, an 18-mer would be presented; on the other hand, if two beads coupled to two oligomers were used to bracket or identify the region, a 9-mer in each case would be sufficient, and use of 18-mers on both sides would considerably enhance the reliability of locus identification. There is, of course, no need that the oligomers be of the same length. For further identification, additional oligomers could be coupled proximal to the region to be interrogated. Most commonly, two identification probes are employed. There is no theoretical reason, however, why a multiplicity of identification probes could not be used cumulatively in the environment of the region to be assessed. The larger the number of bases included in the probes, the more specificity is conferred; however, as indicated above, a total of 18 nucleotides appears sufficient to interrogate most genomes; interrogation of simpler samples may well require less.

While the particulate labels or “beads” are typically coupled directly to oligomers for interaction with the target nucleic acids, in some aspects, the bead may be coupled to a specific binding partner for a detection moiety coupled to an assay probe. “Specific binding partner” refers to a member of a specific binding pair such as biotin/avidin, ligand/receptor, or antibody/antigen.

Thus, the beads may be utilized as labels for “identification probes” which pinpoint the region of the nucleic acid to be interrogated or may be coupled to “assay probes” which are employed to assess the variable region identified by the “identification probes.” While identification probes comprise nucleotide oligomers; assay probes may comprise oligomers or other members of specific binding pairs.

In those embodiments where the identification probes are used to bracket a region of nucleic acid to be interrogated, and where it is desirable to be able to observe the particulate labels or beads coupled to the identification probes individually, the spacing must be sufficient to accommodate the dimension of the beads. Typically, in observing the beads by microscopy, the dimensions appear to “swell” so that the spacing should be sufficient to accommodate the apparent swelling. For example, beads with a diameter of about 100 nm may appear to swell to approximately 200 nm. If the beads are to observed independently, they should be spaced on the target nucleic acid at a distance of approximately at least 250 nm—estimating 100 nm radius for each bead and a 50 nm gap between them. Assuming the spacing of the bases on a typical nucleic acid is of the order of 3.4 Å or 0.34 nm, the 250 nm spacing would represent about 735 bases. Similarly, if one assumes an apparent diameter of 400 nm or radius of 200 nm, a similar calculation would yield a spacing of approximately 1,500 bases. Analogous calculations can be made depending on the diameter of the beads used for labeling and the magnitude of the apparent swelling to result in potential overlap of the beads.

Methods That Employ Identification Probes Only

The methods of the invention find a wide variety of applications in forensics, diagnosis, and the like. For example, these methods are useful to detect infectious organisms. In this embodiment, as well as in others of similar nature, the use simply of identification probes is sufficient. The appearance of one or more beads, preferably two beads, at a particular location serves to verify that the target sequence is present.

The genomes of numerous bacterial, fungal, and viral species have been sequenced, and many more are being catalogued each year. From these data, it is possible to design identification probes that are diagnostic of the infectious agent. By requiring two beads included in two such probes to become associated, the specificity of detection is greatly enhanced, which is important for assays that have the sensitivity to detect extremely small amounts of the agent. Since the number of potential probes is in the thousands, it is advantageous to create particles that comprise a 1 μm bead to which a 0.5 μm bead is attached (covalently or by some appropriately strong non-covalent interaction). With 100 varieties of each type of bead, the number of distinguishable composite particles is 10,000.

Thus, diagnostic methods which simply employ identification probes which bind specifically to targets to be tested for in a nucleic acid-containing sample can readily be multiplexed by employing mixtures of identification probes intended for a multiplicity of analyte sequences to be detected. By employing beads in these identification probes with a multiplicity of hues, large numbers of analyte sequences can be detected in a single sample. The sample treated with the multiplicity of identification probes of various hues is observed microscopically for association of pairs of identification probes of known hue in association with each other in the microscope observation field. Similarly, a standard set of typing reagents can be applied to specimens which might contain one of numerous different identifying features. The direct assay of the specimen against all the typing reagents in one reaction simplifies and accelerates the readout. Internal calibration controls are also readily incorporated into the assay by virtue of the large number of distinguishable tags.

To further simplify the analysis, in one embodiment, the particles are constructed to be denser than water, e.g., by inclusion of small amounts of silica within the bead. After incubation with the specimen to be analyzed, with beads maintained in solution by shaking or sonication, the beads are allowed to settled to the bottom of the incubation chamber. Focusing a microscope solely on the bottom of the chamber to observe the hybridized identification probe reduces noise from the specimen debris and other assay reagents, and concentrates the beads for faster readout.

In an additional application, the presence of two beads with specific hues can be used to identify a synthetic nucleic acid used as a tag to address a member of a specific binding pair, such as an antibody. In this application, the specific binding pair member is coupled to a nucleic acid of sufficient length to accommodate a pair of identification oligomers coupled to beads. If two beads coupled to identification probes are to be distinguished, the tag must be of sufficient length to accommodate and distinguish the beads. The length of the tag will thus be dependent on the diameter of the beads employed.

For intracellular determinations, the tag length may be sufficiently long—e.g., about 1,500 bases as an illustration, that permeability becomes a problem; nucleic acid fragments which contain overlapping complementary termini may be used which self-assemble in the cell. Typically, the member of the specific binding pair may be an immunoglobulin or fragment or recombinant single-chain form thereof and the nucleic acid tag will be a single-stranded form of RNA, DNA, peptide nucleic acid, or other substance falling within the foregoing definition of “nucleic acid.”

Because a variety of probes may be labeled with a multiplicity of differently hued beads, a large number of different specific binding pair members can be used simultaneously to detect a multiplicity of analytes.

The principle of identifying nucleic acid targets with identification probes coupled to beads can be applied to trace the activity of introduced nucleic acids, such as a library of antisense constructs. Thus, expression vectors may include identification probes as portions of double-stranded regions irrelevant to expression. The effects of introduction of the labeled expression vector into the cell is then correlated with observation of the identification probes and the nature of the effect of the expressed sequence correlated with the relevant label. Although single copies of a nucleic acid are detectable by microscopy (Femino, A. M., et al., Science (1998) 280:585-590), transcription of the DNA into multiple RNA molecules improves reliability of detection.

Kits to carry out the foregoing method will typically contain the appropriate identification probes for the target nucleic acids to be assessed. For multiple determinations, the multiplicity of probes may be supplied as a mixture or in arbitrarily divided containers. Instructions for conducting the assay will also be included.

Methods That Employ Identification Probes and Assay Probes or Reagents

Turning now to the examples of invention methods which employ additional assay features besides the identification probes, although single nucleotide or other polymorphisms are suitable for use in forensic DNA applications, a more commonly detected polymorphism in such work relates to measuring the number of copies of a “repeat” in a segment of DNA present at a given locus, which is reflected in a change in the length of a given restriction fragment. An analogous measurement constitutes one aspect of the invention. For example, often measured is the length of an inserted Alu repeat sequence, the “monomer” of which is an approximately 300 base pair “junk” DNA sequence that represents about 5% of the human genome. Various individuals are characterized by a different number of tandem copies of the Alu sequence at characteristic portions of the genome. In the invention method, rather than generating fragments by treating with restriction enzymes targeting flanking sequences and ascertaining the size of the generated fragments, the characteristic region of the target is identified by establishing its boundaries using particulate beads and then measuring the intensity of a label whose intensity is dependent on the number of repeats in the segment. Suitable flanking sequences can be readily chosen now that the human genome sequence has been fully sequenced.

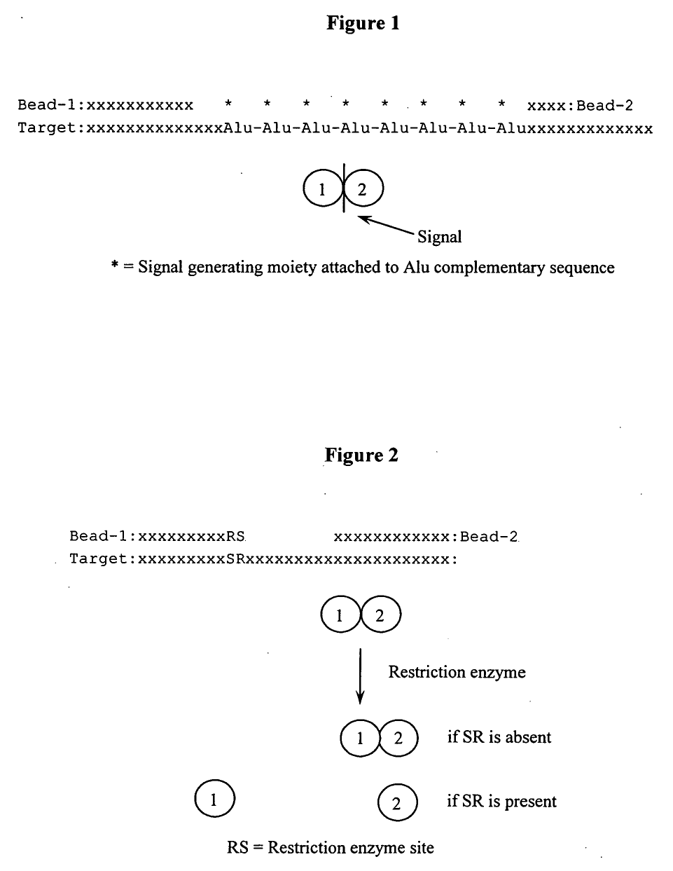

This is illustrated in FIG. 1. As shown, the position of a repeat such as the Alu repeat is bracketed by the labeled beads, i.e., the identification probes, thus establishing the location of the desired segment. The beads will be associated due to the proximity of binding locations on the target. The individual Alu repeats are concurrently labeled by hybridization or other coupling with a fluorescent complement, represented in FIG. 1 by an asterisk (*); the number of fluorescent labels will be proportional to the number of Alu repeats and thus the intensity of the signal from the fluorescent labels will be proportional thereto. The target DNA can be treated with the fluorescent label before, during or after labeling with the particulate labels. As shown in FIG. 1, the viewer will see Beads 1 and 2 in association, with an intervening fluorescent signal generated by the fluorescent label visible between these labels. As noted above, while single-stranded nucleic acids are preferable so that complementary oligonucleotides can be used as coupling reagents in the particulate labels, triplex formation could also be used in this embodiment of the invention. Also, while certainly less desirable, a single bead-coupled oligomer as an identification probe may be used to mark the location of the repeat.

A multiplicity of such polymorphisms can be simultaneously determined by employing multiple identification probes with particulate labels each of different hues so that each individual location to be tested for repeats can be characterized. Again, at least one of the upstream and downstream labels represented by Beads 1 and 2, and preferably both, are created so that a pair of bracketing labels is available for each of the multiplicity of nucleic acids to be tested. To perform the method of the invention with the multiplicity, a mixture of the target nucleic acids to be tested for repeats is treated with a mixture of particulate labels wherein the mixture of particulate labels contains an appropriately derivatized Bead 1 and/or Bead 2, preferably both, for fixing the upstream and downstream boundaries of the repeat. Again, by displaying the reaction mixture on a microscope slide or otherwise so that each individual pair of repeat-flanking beads can be identified as a separate point in space, the various pairs associated with the various targets can be identified. By making each pair destined for binding to each target locus different in hue, the identity of the target nucleic acid locus being observed is immediately readable. The signal generating moieties which bind specifically to the repeat may be the same or different with respect to the various test nucleic acids; the signal generating moiety may be the same for all repeats or different according to the preference of the practitioner. Of course, the signal generating moieties destined for the repeats must be coupled to suitable oligonucleotides or other moieties that are specific for the nature of the repeats in question. Individual intensities of individual pairs can then be observed. Since commercially available solid state light detectors (CCDs) on digital microscopes are quite linear, the quantized intensities arising from different numbers of repeats are readily measured.

Kits for performing the foregoing methods will include any required identification probes and assay probes which comprise the oligomers complementary to the repeating sequences themselves linked to an observable light-emitting label.

Another aspect of the invention is determination of the presence or absence of a SNP in a target nucleic acid as illustrated in FIG. 2. As shown, the assay also takes advantage of identification probes to associate bead labels to target nucleic acids by specific complementarity of these probes. The complementarity can be supplied by any oligomer as defined above where the sequence of attached bases is specifically complementary to the sequence of the target. In this aspect of the invention, it is preferred to use single-stranded DNA in view of the nature of the activity of the restriction enzymes that will be used as assay tools.

As shown in FIG. 2, a first particulate label designated “Bead 1” is coupled to an oligonucleotide which contains a restriction site haploid (RS) designed to be complementary to a stretch of target DNA (illustrated by genomic DNA) which contains the site of a putative SNP. In a sense, this reagent is both an identification probe and an assay probe as it will serve both functions. The SNP is characterized by, in one type of allele, the complementary strand to the restriction site haploid; alternative alleles of the SNP are mutated so that the complementary restriction site haploid is no longer present. As used herein, “restriction site haploid” refers to the single-stranded form of a restriction site, which, when hybridized to its complement, generates a restriction site which will be cleaved by a suitable restriction enzyme. In order to form this restriction site, therefore, the complementary haploids must be hybridized to form double-stranded DNA.

As shown in FIG. 2, the target DNA potentially contains the complement, labeled SR, at such a position that formation of double-stranded nucleic acid will generate a susceptible restriction site where this allele is present but will fail to do so when SR in the target is mutated. Adjacent to the location of the complementation of the first oligonucleotide, a second oligonucleotide coupled to a second particulate label (Bead 2) is bound to the target. The first and second oligonucleotides may essentially be contiguous, allowing only enough space for diffusion of the restriction enzyme into the space (typically 50-500 base pairs); the locations of the complementation must be sufficiently proximal that Bead 1 and Bead 2 are visible in association with each other (typically less than 1500 base pairs). Of course, the probes may be reversed—i.e., either the Bead 1 or Bead 2 oligomer may contain the restriction site haploid.

The target nucleic acid is then treated with the first and second oligomer which will align with the target and be bound thereto as shown. Binding to the target results in the physical proximity of Bead 1 and Bead 2 which are then viewed as associated, typically using a microscope display. The association will, however, be destroyed in the presence of a restriction enzyme specific for the restriction site if the allele containing the restriction site haploid complementary to the RS haploid in the oligomer is present in the target; if the mutated form of the allele is present, the association will remain intact. Restriction enzymes are attractive in this regard for their sequence specificity and the mild conditions used to cleave the DNA. This method is superior to base specific chemical cleavage methods such that described by Gogos, et al., Nucl. Acid Res. (1990) 18:6807-6818.

As with the previously described method to assess the number of repeats, it is evident that by virtue of the ability to provide a wide variety of hues on the particulate labels as described in the above-referenced PCT publication, it will be possible to assess simultaneously a multiplicity of potential SNPs. Thus, oligomers can be designed to bind to a multiplicity of locations of potential SNPs in target DNA and labeled with alternative hues, typically generated by varying the ratio of fluorophores or other signal generating moieties. The multiplicity of bead associations can then be detected in the viewing field of a microscope slide. The location of the individual SNPs is ascertainable by virtue of the characteristic hues of the first and second particulate labels associated with a particular segment of target DNA.

Thus, a multiplicity of single-stranded DNAs may be mixed with a multiplicity of particulate labels. The mixture of particulate labels will contain a suitable pair for each single-stranded DNA in the mixture to be tested. Because it is possible to provide a large number of hues for the particulate labels, it is possible to distinguish, upon observation, the association of appropriate pairs and to identify the target DNA to which each pair is bound. Thus, for example, the particulate label corresponding to Bead 1 and Bead 2 which will target test strand or locus No. 1 may be red; Bead 1 and Bead 2 which target single-stranded target locus 2 may be blue, and so forth. It is preferred, but not necessary, that Bead 1 and Bead 2 be of different hues because their association can be readily identified as the creation of a new hue as opposed to measurement of intensity of emission. If the beads are large enough to be resolved individually, this factor is irrelevant. By displaying the sets of paired beads on a microscope slide or in another appropriate configuration for microscopic observation, including flow cytometry for example, the association of Bead 1 and Bead 2 can be determined and each pairing ascertained as a different point in space. The mixture can be treated sequentially or simultaneously with appropriate restriction enzymes to determine which pairs of Beads 1 and 2 have been associated and which remain in association with each other. Since the beads are more than sufficiently bright to enable detection of single molecules, and are readily multiplexed, a large number of assays can be conducted concurrently at very high sensitivity, which is useful in instances such as forensic DNA testing where quantities of material are severely limited and processing steps introduce opportunities for systematic error arising from contaminants.

Kits for carrying out the foregoing method will contain the combination identification/assay probe which contains the restriction site haploid as well as the identification probe for proximal binding to the site to be tested. Optionally, the kit may also contain the appropriate restriction enzyme. Instructions for performing the assay will also be included.

Of course, the above two approaches described may be combined whereby, the restriction site complementary to the allele to be determined for a SNP is included in one of the oligomers attached to a particulate label used to bracket a tandem repeat locus. Thus, in addition to observing the intensity of the fluorescent label contained within the position fixation beads, the method will include the further step of treating the preparation with the restriction enzymes to detect the dissociation (or remaining association) of the two labels. While it is preferred that this step be carried out subsequent to detecting the intensity of fluorescence characterizing the repeat, this step may also be conducted prior to measuring the intensity of the fluorescence.

Thus, by employing particulate labels to identify specified portions of a target, and, if desired, multiple particulate labels to identify a multiplicity of locations at a target, both the presence or absence of an allele containing a restriction site and the characteristic length of a repeated tandem sequence can be obtained directly on target nucleic acids viewed microscopically. Because microscopic viewing is employed, it is generally unnecessary to amplify the target nucleic acid (although amplification may be performed if desired to increase the density of bead pairs and reduce adventitious non-specific binding to other DNA sites, although the requirement for both upstream and downstream probes to become independently associated with the target reduces non-specific binding by roughly the square root of a single probe value). In the embodiments of the invention where the presence or absence of a restriction site at a particular location is determined, it is preferred to utilize a single-stranded nucleic acid as the target; in the case of determining the length of a tandem repeat, single-stranded targets are also preferred; however, formation of triplexes with double-stranded targets is also within the scope of the invention and desirable in some cases. In all instances, multiple samples can be examined simultaneously by virtue of the availability of a multiplicity of particulate labels for identifying the specific location in the target nucleic acid from which the assessment will be taken.

Another alternative for detection of a SNP also takes advantage of identifying a locus using beads attached to identification probes to define the locus, thereby enabling use of probes with higher differentials in binding energy at a given site but low overall specificity within the genome. In one form of this approach, it is possible to use a labeled oligomer, approximately a 5-mer as an assay probe; in the case of a 5-mer, a single mismatch represents a sufficient differential in free energy of binding that a single mismatch will lead to a readily detectable lack of binding of the probe. The probe is, itself, labeled with a fluorophore, such as a quantum dot. The 5-mer is used merely as an example, any length probe with an advantage in differential can be used—e.g., a 4-mer or 6-mer or 7-mer. As the length increases, the differential diminishes and much of the advantage is lost. Use of peptide nucleic acids (PNA) in particular allows use of shorter probes since the binding energy per base is larger, thus providing a greater relative difference in the case of a perfectly matched 4-mer versus one with a single base mismatch.

In theory, such an assay probe could be used alone to detect the presence or absence of a polymorphism where the assay probe is designed to contain only the sequence characterizing the referent locus—e.g., a 5-nucleotide sequence which contains the referent base. The presence of a SNP representing a single base mismatch will result in failure of the probe to bind and thus the failure of the probe to hybridize effectively to the target. However, this approach cannot usefully be applied to a complex target, such as a restriction digest of an entire genome, since there would be multiple “hits” with respect to such a short sequence. The problem of insufficient specificity could be avoided by utilization of specific primers and PCR or other form of amplification of a desired portion of the target or it could be resolved by using a probe with a much longer sequence. However, the first solution involves the problems inherent in amplification and the latter solution is disadvantageous because the energy differential resulting from a single base mismatch over a longer probe—typically an 18-mer—is insufficient to provide reliable results. It is difficult to find conditions where a conspicuous binding/non-binding event can be observed. (The free energy changes associated with various types of mismatch in short oligos and their effect on binding off-rates have been measured by surface plasmon resonance and the results confirm that a 2 base mismatch is readily detectable (Persson, et al., Anal. Biochem. (1997) 246:34-44). Peptide nucleic acids, which have a higher free energy of binding, may be even shorter.)

The present invention solves the problem of lack of specificity by supplying oligomers coupled to observable beads as identification probes, in a manner analogous to that described above with respect to bracketing tandem repeats. Thus, an oligomer binding upstream of the 5-nucleotide region containing the base to be interrogated and an alternate oligomer which binds downstream thereof, each coupled to an observable bead, permits the relevant region to be bracketed. Only assay probe binding to the 5-mer bracketed by the beads is then observed, as described above.

This approach suffers only from the disadvantage that a specific assay probe must be designed for each targeted SNP. It would be advantageous to modify this method so as to permit a minimum number of assay probes to be useable over a full range of possible loci. This can be accomplished if only the base to be interrogated must be varied; under these circumstances, only four different assay probes would be required to interrogate all possible targets.

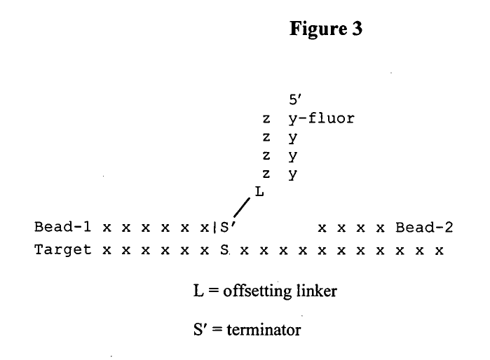

This modified method is illustrated in FIG. 3. As shown, the upstream and downstream oligomers coupled to beads used as identification probes bracket the base to be interrogated, symbolized by “S.” An assay probe contains the complement to one embodiment of S in the form of an extension terminator; a mismatch with S will result in lack of incorporation of the terminator and thus incorporation of the assay probe will not take place when the complex is treated with polymerase. If the terminator complements S, the terminator, and thus the assay probe, will be incorporated. The assay probe contains the interrogating terminator coupled through a linker that offsets the remainder of the probe from the target sequence; such linkers can be found, for example, in U.S. Pat. No. 5,916,750, incorporated herein by reference. This linker is, in one embodiment, attached to an additional sequence, shown as a 4-mer, but which could be of any length, of known base sequence. More generically, the linker may be coupled to a member of a different specific binding pair, such as biotin/avidin or antigen/antibody, or directly to a fluorophore. The bound or unbound assay probe containing a specific binding partner is then, itself, probed with a detection probe, which is a labeled complement of the extended terminator oligomer as shown or the complementary member of an alternative specific binding pair.

In more detail, a first oligomer coupled to an observable bead, as an identification probe, is hybridized so as to serve as a primer for an extension reaction in the presence of polymerase. This first oligomer/primer extends to a position immediately upstream of the base to be interrogated on the target strand. Optionally and preferably, a second oligomer, also coupled to a bead (another identification probe) is hybridized proximal to the interrogated base so as to bracket this position. This confers additional specificity by requiring additional sequences that must match the target locus. An assay probe, such as a terminator oligomer, is included in the reaction mixture; the termination oligomer contains, at the 5′ terminus, a nucleotide such as a dideoxynucleotide, which will be incorporated as an extension of the first oligomer/primer, if it is complementary to the interrogated base, but cannot result in further extension. This terminator is coupled to an offsetting spacer, or linker, as described above, which is, in turn, either extended by a nucleotide sequence or by another specific binding pair member as a means to bind a detection probe bearing a fluorophore. The assay probe could also be extended by a fluorophore-containing moiety. The extension comprising a means to bind a detection probe can then itself be coupled to a labeled complementary oligomer or other specific binding partner used to detect the assay probe. Because of the presence of the offsetting spacer, this assay probe is sometimes referred to as a “swivel.”

Thus, this method involves, in addition to the identification probes which serve to identify the interrogated site, either an assay probe (which contains a terminator coupled to an offsetting spacer coupled to a member of a specific binding pair) and a detection probe which is a fluorophore-labeled complement to the specific binding pair of the assay probe, or an assay probe which comprises a terminator and an offset fluorophore. The entire assembly is viewed under a microscope where the proximity of the labels coupled to the identification probes and the label coupled to the assay or detection probe will be observed in proximity in the event the terminator is complementary to the interrogated base.

In a manner similar to that described in regard to the methods for detection using identification probes only, to methods for measuring the length of repetitive element-containing sequences, and for detecting SNPs comprising restriction enzyme sites, this method for detecting SNPs may also be multiplexed. As in the previous cases, the identification probes are supplied directed to a multiplicity of loci in a multiplicity of hues. The composition of the assay probes may be identical for all sites to be tested that contain the same embodiment of the base to be tested, or may be different; it is possible to use only four assay probes of the type described above to interrogate a multiplicity of loci. The method is susceptible to multiplexing because of the ability to observe the binding of the assay probe (either directly or by virtue of an additional detection probe) as associated with a particular locus identified by the identification probes.

Kits for performing the foregoing methods will contain the one or more identification probes for each locus to be tested and an appropriate assay probe which will be supplied either as a labeled approximately 5-mer or the more complex assay probes which employ a terminator for primer extension. Optionally, the DNA polymerase required may also be supplied. If the assay probe does not itself contain a fluorophore, an additional detection probe will be included in the kit.

Applications

One application of the invention method relates to genotyping bacterial or viral nucleic acids.

Bacterial or viral species can be typed based on conserved sequences using pairs of identification probes according to the invention; subtypes can be further visualized by the same approach as that used for SNP detection.

In another application, HLA typing can be accomplished, beginning with conserved sequences using pairs of identification probes and subtyping by the methods used for SNP detection. It is advantageous to measure the HLA phenotype on mRNA, which is more abundant than the encoding gene.

In all of these applications, the high degree of multiplexing provided by the methods of the invention has the practical utility of allowing one batch of reagents to be used for a wide range of assays. In any given instance, only a few of the probes will successfully form pairs, but since the cost of reagents is minimal due to the microscopic scale of the assays, it is more efficient to assay for everything at once than to perform sequential tests based on a hierarchical phylogenetic typing scheme.

The following examples are intended to illustrate but not to limit the invention.

### Preparation A

### Feasibility of Hybridization Detection—Bead-Coupled Oligomers

Conjugation of DNA to beads followed by hybridization and observation of coupled beads is illustrated in a test system using complementary 28-mers. Each 28-mer contains a spacer of seven nucleotides at the 5′ end which is coupled to biotin, thus permitting a capture of the oligomer onto avidin-coated beads. The remaining 21 nucleotides of each oligomer are complementary. The estimated Tm of the double-stranded region obtained when the oligomers are hybridized is 59° C. The first oligomer is labeled with a red avidin-coated bead and the second oligonucleotide with a green avidin-coated bead. Avidin-coated beads in both red and green fluors are available in size ranges from 0.2-1.1 μm from Molecular Probes, Seattle, Wash.

To label each oligomer, 20 μL of the red or green bead stock (1% solid content) are mixed with 40 μL 20 μM HPLC purified biotinylated oligonucleotide and the volume adjusted to 800 μL with Buffer A (10 mM sodium phosphate, 0.1 M NaCl, pH 7.0 with 3.7 mM lithium dodecyl sulfate). The binding reaction is incubated in a 1.5 mL microcentrifuge tube on an end-over-end rotator for 12 h at 23° C. Unbound oligonucleotides are removed by washing the beads by centrifugation three times in 800 μL Buffer A at 10,000 rpm for 8 min. The beads are subsequently resuspended to a final concentration of 0.02% solids in desired buffer containing 0.04% NaN₃. Beads can be stored in the dark at 4° C. for more than 4 weeks without any loss of binding activity.

To hybridize the oligomers, the first oligomer labeled with a red bead and the second oligomer labeled with a green bead are mixed (final concentration 0.01-0.02% solids) and put on a rotator. At desired time points (0, 4, 8, 12 and 24 h), 5 μL samples are taken from the, hybridization reaction and applied to microscope glass slides, either directly or by using a Cytospin centrifuge (ThermoShandon). The specimen is overlaid with mounting medium (Prolong, Molecular Probes) and covered with a cover slip prior to microscope analysis.

Images of beads applied to microscope slides are captured with a fluorescence microscope equipped with a high resolution charge coupled device camera (DeltaVision from Applied Precision (Seattle, Wash.)). A 0.3 mm portion of the slide is photographed in 86 μm×86 μm square tiles. When equally sized beads were used, only one Z-section is collected. For different sized beads, the number of Z-sections is chosen so that every type of bead is in focus in at least one Z-plane. Each image file contains more than 500 of each bead. The number of each bead type and the occurrences of bead to bead binding are counted using a modified version of ImageJ software available from National Institutes of Health (NIH). Results are further processed in Microsoft Excel for statistical analysis and graphical presentation.

Typically, at least 80% of the beads are in red/green pairs. Thus, even short complementary DNA oligos (attached to red or green beads respectively) can hybridize without significant hindrance from the beads to which they are attached, with a “spacer” arm of only seven nucleotides. Negligible numbers of red/red or green/green pairs are counted.

### EXAMPLE 1

### Determination of Variable Number Tandem Repeat (VNTR) Polymorphisms

To identify 16 loci, beads are prepared using four intensity levels for each of blue and red fluors (providing 16 ratios, each representing a hue, in the manner described in WO 00/14545 referenced above). For each locus, two beads of the same hue are employed; one coupled to the 5′ end of a probe designed to bind one border of the locus, and a second bead of the same hue coupled to a oligomer at the 3′ end which is designed to hybridize to the other border of the locus.

Genomic DNA is denatured and sheared and mixed with the 32 oligomers prepared as described. Each locus is then labeled at its borders with a bead of characteristic hue so that the locus can be identified. The mixture is maintained under hybridization conditions and washed, resulting in a multiplicity of DNA segments containing tandem repeats labeled at the borders with identifying beads.

The mixture of DNA fragments is then placed in a hybridization mixture with oligomers coupled to a green fluor; the oligomers are designed to hybridize to the repeating segments of the various loci. The hybridization mixture is washed and applied to microscope slides as beads of 500 nm in diameter are used, individual beads can be resolved according to this technique.

The mixture is observed and recorded as described in Example 1. Each locus can be identified and observed as a single pair of beads of the same hue which emits green fluorescence in an intensity corresponding to the number of repeats between the borders.

Approximately 2,500 pairs are visualized in a single frame of the digital microscope, which takes ˜1 second to record. Thus, over 100 replicates of the assay for each locus is conducted, providing adequate statistics to identify both polymorphic alleles at each site.

Internal calibration of the Alu reaction is also feasible by arranging for the detection probe to include a sequence that hybridizes to a sequence immobilized at known concentration on the bead. Preferentially, that calibration sequence is on a 0.5 μm bead covalently attached to the 1 μm bead used to demarcate the upstream or downstream sequence on the DNA.

### EXAMPLE 2

### Application of VNTR Polymorphism Determination to Huntington's Disease Diagnosis

Huntington's Disease is characterized by tandem repetition of codons for glutamine in the Huntington protein. While normal individuals have 9-35 copies of glutamine at this point in the protein sequence, Huntington's Disease patients have 36 to 121 copies, with age of incidence proportional to extent of extra copies (over 50 copies generally results in young adult onset).

Genomic DNA from subjects to be analyzed for susceptibility to Huntington's Disease are denatured and subjected to the procedures set forth in Example 1. A 27-mer oligo labeled with green fluor containing nine copies of the complement to the codon encoding glutamine is used as the fluorescent probe. Normal individuals will thus provide samples which exhibit a green fluorescence intensity proportional to the binding of 1-4 copies of the probe while susceptible individuals will show higher intensities corresponding to the binding of 5 or more copies.

It will be noted that because of the specificity designed into the beads which are designed to hybridize upstream and downstream of the poly-glutamine codon stretch, it is not necessary first to isolate the gene encoding Huntington protein; only hybridization to the poly-glutamine in the relevant portion of the genomic DNA will be recorded.

### EXAMPLE 3

### Detection of a SNP Using Key-Swivel Technology

For high volume SNP analysis, polymorphisms are selected in which the variable base is G. The data are scored as G or not-G.

A quantum dot is used as the fluor on a key detecting G, as illustrated in FIG. 3. Because the quantum dot emission bandwidth is small, this choice allows three colors of organic dye fluors to be used to define the hue of the beads. Each bead is created by covalently attaching a 500 nm particle to a 1 μm diameter particle. With three colors, each readable in five intensity levels, 125 types of 500 nm particle and 125 types of 1 μm particle can be made, resulting in 15,625 distinct hues available for Bead 1 and corresponding numbers of Bead 2 types. A 5′ and 3′ probe for the gene of interest is attached respectively to the same hue of Bead 1 and 2. Hybridization to sheared and denatured genomic DNA results in capture of two beads of the same hue in close proximity. Background non-specific binding that affects one bead is unlikely to affect the other bead of the pair at the same site. The SNP site is then interrogated by using polymerase to add a single dideoxycytosine, which will be incorporated only if the target SNP site comprises a guanosine. The incorporated dideoxy terminator also includes a moiety extending out of the plane of the phosphodiester backbone. It is well established that biotin on a 12-18 carbon spacer arm attached to the nucleoside does not interfere with enzymatic incorporation. Biotin, then, provides one example of a lock, with avidin as a key. Many other biospecific pairs can also be used, including short oligonucleotides. The presence of a key hybridizing to the lock at the interface of the Bead 1 and Bead 2 is indicative of G being present. Approximately 1,000 such pairs are visualized in a single frame of the digital microscope, which takes ˜1 second to record. At 60-fold redundancy to achieve reliable reading, a full panel of 15,000 SNP loci is readable in 15 minutes.

Alternatively, all four nucleotides are included in the reaction, each with a different lock moiety to which a uniquely readable key can be bound. In this way, large keys, such as quantum dots, can be introduced into the assay, which would not be feasible with direct incorporation of the fluor on the single dideoxynucleotide.

Suitable DNA is obtained from whole blood by one of the various methods known in the art of DNA analysis, such as the Extract-N-Amp Blood PCR kit sold by Sigma (St. Louis, Mo.), or the QuickExtract kit sold by Epicentre (Madison, Wis.). Random DNAse cleavage is used to produce fragments averaging 5 kilobases. A small number of PCR rounds (˜5) is useful to increase the density of bead pairs, but is not strictly necessary.

### EXAMPLE 4

### Multiplexed Antibody Staining

Antibodies directed to a substance whose location within a cell is to be determined are coupled to a fragment of single-stranded DNA (tag DNA) of sufficient length to accommodate a pair of distinguishable identification probes coupled to fluorescent beads, 1,500 base pairs typically being sufficient. Two such beads, coupled to fluorophores of known hue are conjugated to single-stranded oligomers DNA complementary to the 5′ and 3′ ends of the tag DNA. The identification probes which include these beads are administered to the cell along with the DNA tagged antibody. The antibody coupled to the analyte present intracellularly can then be detected by means of the pair of beads and thus the location of the target analyte is ascertained.

Because a multiplicity of hues can be generated for use in the identification probes, a multiplicity of antibodies to a multiplicity of targets may be employed simultaneously to note the position of a desired number of analytes.

Alternatively, in order to improve the permeability of the components of the tag DNA, the tag may be assembled intracellularly from a multiplicity of single-stranded DNA's with overlapping complementary termini. Thus, the antibody is bound to a 50 base segment; a number of 50 base oligomers with overlapping regions are supplied until a desired length is reached. The terminal segment will have an uncomplemented extension to accommodate the suitable identification probe.

Alternatively, the overlapping fragments used to extend the tags may themselves include labeled nucleotides which generate a multiplicity of hues.

1. A method to identify a desired region of a target nucleic acid to be targeted for observation, which method comprises
contacting said nucleic acid with first and second identification probes, which probes comprise first and second oligomers specific for the upstream and downstream sequences bracketing said region respectively,
wherein said first oligomer is coupled to a first particulate label and said second oligomer is coupled to a second particulate label and wherein said particulate labels are observable by microscopy.

2. The method of claim 1, wherein said first and second particulate labels comprise fluorophores.

3. The method of claim 1, wherein said first and second labels are different.

4. The method of claim 1, wherein said first and second oligomers are peptide nucleic acids.

5. The method of claim 1, wherein said target nucleic acid is single-stranded and said first and second oligomers are complementary to the upstream and downstream sequences bracketing said region.

6. The method of claim 1, wherein said target nucleic acid is double-stranded and said first and second oligomers form triplexes with said upstream and downstream sequences bracketing said region.

7. The method of claim 1, which is performed simultaneously on a multiplicity of target nucleic acids using a multiplicity of identification probes having particulate labels of differing hues coupled to oligomers comprising sequences complementary to a multiplicity of said upstream and downstream sequences bracketing a multiplicity of such regions.

8. A method to detect the presence of a target nucleic acid of known sequence, which method comprises
contacting said nucleic acid with at least first and second identification probes, which probes comprise first and second oligomers specific for proximal nucleotide sequences of said nucleic acid,
wherein said first oligomer is coupled to a first particulate label and said second oligomer is coupled to a second particulate label and wherein said particulate labels are observable by microscopy.

9. The method of claim 8, wherein said first and second particulate labels comprise fluorophores.

10. The method of claim 8, wherein said first and second labels are the same.

11. The method of claim 8, wherein said first and second oligomers are peptide nucleic acids.

12. The method of claim 8, wherein said target nucleic acid is single-stranded and said first and second oligomers are complementary to the upstream and downstream sequences bracketing said region.

13. The method of claim 8, wherein said target nucleic acid is double-stranded and said first and second oligomers form triplexes with said upstream and downstream sequences bracketing said region.

14. The method of claim 8, which is performed simultaneously on a multiplicity of target nucleic acids, using a multiplicity of identification probes having particulate labels of differing hues for each known sequence targeted coupled to oligomers with different specificities according to the sequences targeted.

15. The method of claim 8, wherein said target nucleic acid of known sequence is derived from an organism.

16. The method of claim 15, wherein the organism is an infectious agent.

17. The method of claim 15, wherein the organism is a human subject.

18. A composition which comprises a target nucleic acid, said nucleic acid comprising a region bracketed by sequences formed by binding to a first oligomer and a second oligomer, said first oligomer coupled to a first particulate label and the second oligomer coupled to a second particulate label wherein said first and second particulate labels are observable by microscopy.

19. The composition of claim 18, wherein said first and second oligomers are peptide nucleic acids.

20. The composition of claim 18, wherein said target nucleic acid is single-stranded and said region is bracketed by double-stranded sequences formed by hybridizing to said first and second oligomers.

21. The composition of claim 18, wherein said target nucleic acid is double-stranded and comprises a region bracketed by triplex sequences formed by triplexing to said first and second oligomers.

22. The composition of claim 18, wherein said region comprises repetitive elements.

23. The composition of claim 22, wherein said repetitive elements are coupled to signal-generating moieties.

24. The composition of claim 20, wherein said target nucleic acid includes, associated with said bracketed region, at least one assay probe.

25. The composition of claim 24, wherein said assay probe comprises a terminator linked through an offsetting moiety to a fluorophore or to means for coupling a fluorophore.

26. The composition of claim 25, wherein said means for coupling a fluorophore comprises a member of a specific binding pair.

27. The composition of claim 26, wherein said assay probe is coupled to a detection probe, which comprises a fluorophore coupled to the complementary member of the specific binding pair.

28. A method to assess the length of a target nucleic acid segment that is composed of a variable number of repeated sequences which method comprises
coupling a first identification probe comprising a first particulate label coupled to a first oligomer specific for a sequence upstream of said segment and optionally a second identification probe comprising a second particulate label coupled to a second oligomer specific for a sequence downstream of said segment wherein said first and second labels are observable by microscopy;
coupling each repeated sequence in said segment to a signal generating moiety of predetermined intensity; and
observing the total intensity of said signal generating moiety coupled to said first and second particulate label.

29. The method of claim 28, wherein said observing is by means of a wide field microscope.

30. The method of claim 28, wherein said first and second particulate labels are of different hues.

31. The method of claim 28, which is performed simultaneously on a multiplicity of target nucleic acids using a multiplicity of identification probes having particulate labels of differing hues coupled to oligomers with different specificities according to the sequences upstream and downstream of the target nucleic acid segments.

32. The method of claim 28, wherein said target nucleic acid segment is single-stranded and said first and second oligomers are complementary thereto.

33. The method of claim 28, wherein said target nucleic acid segment is double-stranded and said first and second oligomers form triplexes therewith.

34. A method to detect a single nucleotide polymorphism (SNP) at a base that is included in a restriction site haploid, which method comprises
reacting a single-stranded nucleic acid target to be tested for the presence of said SNP with a first identification probe which comprises a first oligonucleotide coupled to a first particulate label, which first oligonucleotide contains a complementary restriction site haploid and is further complementary to the portion of the test nucleic acid that contains said base such that said complementary restriction site haploids together generate a restriction site;
reacting said test nucleic acid with a second identification probe which comprises a second oligonucleotide coupled to a second particulate label, which second nucleotide is complementary to a portion of the nucleic acid target proximal to the restriction site haploid
whereby double-stranded nucleic acid containing a restriction site is obtained when the restriction site haploid in said first oligonucleotide is complementary to a restriction site haploid in the test nucleic acid target;
treating said double-stranded nucleic acid with a restriction enzyme which cleaves at said restriction site; and
observing the association or dissociation of said first and second particulate label,
whereby continued association of said first and second particulate labels indicates the absence of the complement to said first oligomer restriction site haploid and dissociation of said first and second particulate label indicates the presence of the complement to said first oligomer restriction site haploid.

35. The method of claim 34, wherein said first and second particulate labels are of different hues.

36. The method of claim 35, wherein said observing is by means of a wide field microscope.

37. The method of claim 35, which is performed simultaneously on a multiplicity of single-stranded DNA targets and wherein said method comprises contacting said multiplicity of targets with a corresponding number of first and second identification probes having particulate labels of different hues such that the first and second-identification probes for each target comprises a particulate label of different hue from that of the first and second identification probes of the remaining targets in said multiplicity.

38. A method to detect a single nucleotide polymorphism (SNP) which method comprises
providing a reaction mixture that has been prepared by reacting a single-stranded nucleic acid target to be tested with a first identification probe comprising a first oligomer, coupled to a first particulate label, which first oligomer is complementary to said target and has a 3′ terminus which is complementary to the nucleotide immediately upstream of a base to be interrogated and by reacting said target with an assay probe which probe comprises a terminator complementary to one embodiment of said base, coupled to a fluorophore or a means to attach said fluorophore through a linker which offsets said fluorophore or a means to attach said fluorophore; and optionally, by reacting the target nucleic acid with a second identification probe comprising a second oligomer coupled to a second particulate label which is complementary to the target nucleic acid proximal to the base to be interrogated; and
treating said reaction mixture with a polymerase
whereby, if said embodiment of the interrogated base is present, the assay probe is incorporated; and
if necessary, attaching a fluorophore to said means to attach a fluorophore; and
observing the association of said fluorophore with said first particulate label and optionally with said second particulate label, whereby the association of the fluorophore with said label(s) indicates the presence of said embodiment of the interrogated base, and the absence of said association indicates the absence of said embodiment.

39. The method of claim 38, wherein said terminator is a dideoxynucleotide complementary to the embodiment of said base, said means to attach a fluorophore comprises a member of a specific binding pair.

40. The method of claim 39, wherein the members of said specific binding pair are nucleotide sequences of at least four nucleotides.

41. The method of claim 39, wherein said specific binding pair is biotin/avidin, antigen/antibody or receptor/ligand.

42. The method of claim 38, which further comprises coupling said means to attach a fluorophore to a detection probe comprising said fluorophore.

43. The method of claim 42, wherein said detection probe comprises said fluorophore coupled to the complementary member of the specific binding pair.

44. The method of claim 38, wherein said observing is by means of a wide field microscope.

45. A method to detect a SNP in a single-stranded nucleotide target, which method comprises
identifying the locus of a base to be interrogated for the presence or absence of a referent base by hybridizing a first identification probe comprising a first oligomer coupled to a particulate label and optionally a second identification probe comprising a second oligomer coupled to a second particulate label at the border(s) of said locus,
treating said hybridized target with a 5-mer assay probe which is completely complementary to said locus when one embodiment of said base is present; and
observing the presence or absence of said assay probe in association with said labels,
whereby the presence of said assay probe associated with said labels indicates the presence of the embodiment of the base and the absence of said assay probe associated with said labels indicates the absence of said embodiment of the base.

46. The method of claim 45 wherein said observing is by means of a wide field microscope.

47. The method of claim 45, wherein said 5-mer is a peptide nucleic acid.
